SEQUENCE LISTING (1 sequence)

SEQ ID NO: 1 - protein, 1095 aa, Artificial Sequence
  Synthetic Construct
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSASTGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGAASDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLAAALQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEWLGVWGQGTLVTVSSGGGGSSGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLG

(19) United States
(12) Patent Application Publication
(10) Pub. No.: US 2015/0139936 A1
(43) Pub. Date: May 21, 2015

(54) DOSAGE AND ADMINISTRATION OF BISPECIFIC SCFV CONJUGATES IN COMBINATION WITH ANTI-CANCER THERAPEUTICS

(71) Applicant: Merrimack Pharmaceuticals, Inc., Cambridge, MA (US)

(72) Inventors: Sasha Frye, Waltham, MA (US); Charlotte McDonagh, Winchester, MA (US); Victor Moyo, Cambridge, MA (US)

(73) Assignee: MERRIMACK PHARMACEUTICALS, INC., Cambridge, MA (US)

(21) Appl. No.: 14/400,223

(22) PCT Filed: May 13, 2013
(86) PCT No.: PCT/US2013/040785

§ 371 Date: Nov. 10, 2014

Related U.S. Application Data
(60) Provisional application No. 61/726,906, filed on Nov. 15, 2012.
(60) Provisional application No. 61/701,184, filed on Sep. 14, 2012.
(60) Provisional application No. 61/645,892, filed on May 11, 2012.

(51) Int. Cl. A61K 39/395; A61K 31/337; A61K 31/517; C07K 16/28

(52) U.S. Cl. CPC A61K 39/39558; C07K 16/2863; A61K 31/337; A61K 31/517; A61K 2039/507
USPC 424/85.1; 424/136.1; 530/387.3

(57) ABSTRACT

Provided are methods and compositions for clinical treatment of advanced HER2 positive solid tumors cancer using combination therapies comprising bispecific anti-ErbB2/anti-ErbB3 antibodies.

### CROSS-REFERENCE TO RELATED APPLICATIONS

This application claims benefit of priority to U.S. Provisional Application No. 61/645,892, filed May 11, 2012, U.S. Provisional Application No. 61/701,184, filed Sep. 14, 2012, and U.S. Provisional Application No. 61/726,906, filed Nov. 15, 2012, each of which is hereby incorporated by reference.

### BACKGROUND OF THE INVENTION

Despite improvements in cancer therapies and late-stage options, there remains a critical need to optimize established therapies and develop new, promising therapies which prolong patients' lives while maintaining a high quality of life.

In the treatment of cancers, the co-administration of pluralities of anti-cancer drugs (combination therapy) often provides better treatment outcomes than monotherapy. Such outcomes can be subadditive, additive, or superadditive. That is to say that the combined effects of two anti-cancer drugs, each of which provides a quantifiable degree of benefit, can be less than, equal to, or greater than the sum of the benefits of each drug. For example, two drug, each of which when used alone to treat a lethal cancer provides an average one year extension of progression free survival, could together provide a <24 month extension (e.g., an 18 month extension), about a 24 month extension, or a >24 month extension (e.g., a 30 month extension) of progression free survival. Typically, combination therapies for cancer treatment provide significantly subadditive outcomes. Outcomes that are near additive, additive, or superadditive are most desirable, but only occur rarely. In addition, many drugs are known to alter the bioavailability, or otherwise affect the safety profile of other drugs when both drugs are co-administered. As new drugs are first used in combination therapies, unforeseen, hazardous drug-drug interactions may be observed that result in drug-drug interaction-mediated toxicity in the patient.

Thus approaches for safely administering combination therapies comprising administration of ErbB2/ErbB3 heterodimer-targeted agents for cancer treatment, and especially combinations that yield near-additive, additive, or superadditive outcomes are needed.

### SUMMARY OF THE INVENTION

Provided are compositions and methods for treating a cancer in a human patient, the methods comprising administering to the patient a combination of a bi-specific anti-ErbB2/anti-ErbB3 antibody and at least one additional anti-cancer therapeutic, wherein the combination is administered (or is for administration) according to a particular clinical dosage regimen (i.e, at a particular dose amount and according to a specific dosing schedule). In an exemplary embodiment, the patient has a cancer that is a HER2-positive (i.e., in which HER2 is overexpressed) solid tumor. HER2 positivity can be determined by, e.g., fluorescence in situ hybridization (FISH, which detects HER2 gene amplification), chromogenic in situ hybridization (CISH, which also detects HER2 gene amplification), or by immunohistochemistry assays such as HERCEPTEST® (which measures levels of HER2 protein as HER2 negative, HER2 1+, HER2 2+, or HER2 3+). The methods and compositions provided herein are useful for treatment of cancers that are HER2-positive (particularly those that test HER2 2+ or HER2 3+) or FISH OR CISH positive. In one embodiment, the patient has a cancer that is a brain, breast, esophageal, gastric, gastro-esophageal junction, bladder, ovarian, endometrial, or non-small cell lung cancer. In another embodiment, the cancer is a melanoma, a cholangiocarcinoma, a clear cell sarcoma, or a head and neck, prostate, colon, colorectal, lung, pancreatic, salivary gland, liver, skin, brain or renal tumor. In other embodiments, the cancer is squamous cell cancer, small-cell lung cancer, cervical cancer, or thyroid cancer. In still another embodiment, the cancer is not metastatic.

An exemplary bispecific anti-ErbB2/anti-ErbB3 antibody is MM-111 (SEQ ID NO:1). MM-111 and number of bispecific anti-ErbB2/antiErbB3 antibodies suitable for use with the methods and compositions provided herein are described in, e.g., co-pending US patent publication No. 2011-0059076. Suitable bispecific antibodies disclosed therein include A5-HSA-ML3.9, ML3.9-HSA-A5, A5-HSA-B1D2, B1D2-HSA-A5, B12-HSA-B1D2, B1D2-HSA-B12, A5-HSA-F5B6H2, F5B6H2-HSA-A5, H3-HSA-F5B6H2, F5B6H2-HSA-H3, F4-HSA-F5B6H2, F5B6H2-HSA-F4, B1D2-HSA-H3, and H3-HSA-B1D2.

In one aspect is provided methods and compositions for treatment (e.g., safe and effective treatment) of a cancer in a human patient, the method comprising administration to the patient of an effective amount of (a) a bispecific anti-ErbB2/anti-ErbB3 antibody, (b) lapatinib, (c) a taxane that is paclitaxel, and (d) trastuzumab, wherein the treatment comprises a first cycle of administration and at least one subsequent cycle of administration, wherein each cycle of administration spans a period of four weeks, and wherein:

during each of the four weeks of the first cycle,
(a) is administered at a weekly dose of at least about 5 mg/kg (e.g., at least about 5 mg/kg to about 50 mg/kg),
(b) is administered at a daily dose of at least about 750 mg (e.g., at least about 750 mg to about 1,500 mg),
(c) is administered at a weekly dose of at least about 80 mg/m² (e.g., at least about 80 mg/m² to about 150 mg/m²), and
(d) is administered at a weekly dose of at least about 2 mg/kg (e.g., at least about 2 to about 10 mg/kg), and
   during each of the four weeks of each subsequent cycle,
(a) is administered at a weekly dose of 5 (or about 5), 10 (or about 10) or 20 (or about 20) mg/kg (or alternatively a dose in the range of about 5 mg/kg to about 50 mg/kg),
(b) is administered at a daily dose of 750 (or about 750) or 1000 (or about 1000) mg (or alternatively a dose in the range of about 250 mg to about 1,500 mg),
(c) is administered at a weekly dose of 80 (or about 80) mg/m² (or alternatively a dose in the range of about 80 mg/m² to about 150 mg/m²), and
(d) is administered at a weekly dose of 2 (or about 2) mg/kg (or alternatively a dose in the range of about 2 to about 8 mg/kg).

In one embodiment of this aspect, in at least initial dosing during the first cycle of administration, one or more of (a), (b), (c) and (d) is at a loading dose that is greater than corresponding doses of one or more of (a), (b), (c) and (d) administered in each subsequent cycle. In another embodiment, in at least an initial dose of the first cycle, the trastuzumab is administered at a loading dose of 4 (or about 4) mg/kg (e.g., a dose in the range of greater than about 4 mg/kg to about 20 mg/kg). In yet another embodiment, during each week of each cycle, order of administration is: (b) is administered first, (c) is administered second, (d) is administered third, and (a) is administered fourth.

In another aspect is provided methods and compositions for treatment (e.g., effective treatment) of a cancer in a human patient, the method comprising administration to the patient of an effective amount of (a) a bispecific anti-ErbB2/anti-ErbB3 antibody, (e) a taxane that is docetaxel and (d) trastuzumab, wherein the treatment comprises a first cycle of administration and at least one subsequent cycle of administration, wherein each cycle is a period of three weeks, and wherein:

once during the first cycle:
(a) is administered at a dose of at least about 15 mg/kg (e.g., at least about 15 mg/kg to about 80 mg/kg),
(e) is administered at a dose of at least about 75 mg/m² (e.g., at least about 75 mg/m² to about 150 mg/m²), and
(d) is administered at a dose of at least about 6 mg/kg (e.g., at least about 6 to about 12 mg/kg), and
   once during each subsequent cycle:
(a) is administered at a dose of 15 (or about 15), 20 (or about 20) or 40 (or about 40) mg/kg (e.g., a dose in the range of about 5 mg/kg to about 100 mg/kg),
(e) is administered at a dose of 75 (or about 75) mg/m² (e.g., a dose in the range of about 20 mg/m² to about 150 mg/m²), and
(d) is administered at a dose of 6 (or about 6) mg/kg (e.g., a dose in the range of about 5 to about 12 mg/kg).
   In one embodiment of this aspect, in the first cycle, one or more of (a), (e), and (d) is administered at a loading dose that is greater than the corresponding doses of one or more of (a), (e), and (d) administered in each subsequent cycle. In another embodiment, the trastuzumab is administered during the first cycle at a loading dose of 8 (or about 8) mg/kg (e.g., a dose in the range of greater than about 6 mg/kg to about 30 mg/kg). In yet another embodiment, during each three week cycle, order of administration is: (e) is administered first, (d) is administered second, and (a) is administered third.

In one embodiment of either of the preceding aspects, the treatment comprises at least 20 cycles (e.g., 20 to 50 cycles or more).

In one embodiment of either of the preceding aspects, (a) comprises a polypeptide having an amino acid sequence as set forth in SEQ ID NO:1.

In another embodiment of either of the preceding aspects, the patient is pretreated prior to administration of the taxane with at least one dose of an agent (e.g., dexamethasone, diphenhydramine, cimetidine, or ranitidine) that prevents taxane hypersensitivity. In one embodiment, the at least one dose of the agent that prevents hypersensitivity is two 20 mg doses of dexamethasone; one dose of 50 mg of diphenhydramine; one dose of 300 mg of cimetidine; or one dose of 50 mg of ranitidine.

In still another embodiment of either of the preceding aspects, following the treatment the patient undergoes surgery to remove cancerous tissue. In one embodiment, following surgery, the patient receives further treatment with one or more of (a), (b), (c), (d), and (e).

In another aspect, methods and compositions for treating patients that have metastatic or locally advanced (unresectable) HER2-expressing distal esophageal, GE junction or gastric carcinoma, and that have progressed following treatment with front line fluoropyrimidine/platinum with or without trastuzumab is provided. Preferably, the patients are HER2 2+ or HER2 3+ by IHC. In some embodiments, the patients are HER2 2+ and FISH positive. In other embodiments, the patients are HER2 2+ but FISH negative. The patients are treated with a regimen that follows a 4-week treatment cycle with dose administration of MM-111 and trastuzumab once every 7±2 days. The anticancer therapies will be administered by IV infusion in the following order:

a) Paclitaxel, which is administered as an IV infusion over a period of about 60 minutes. Preferably, the infusion is prepared as directed in the paclitaxel package insert and in compliance with any institutional guidelines; and

b) Trastuzumab, which is administered first as a loading dose of about 4 mg/kg over a period of about 90 minutes followed by weekly dosing at about 2 mg/kg over about 30 minutes by IV infusion; and

c) MM-111, which is administered in a first dose over a period of about 90 minutes followed by weekly dosing over about 60 minutes in the absence of infusion-related reactions.

In a preferred embodiment, the Paclitaxel, Trastuzumab, and MM-11 are administered consecutively without any time interval between the administrations of each component of the regimen. In another embodiment, Paclitaxel may be administered for the first 3 weeks of the 4-week treatment cycle followed by 1 week off of paclitaxel therapy.

In another aspect, methods and compositions for treating patients that have metastatic or locally advanced (unresectable) HER2-expressing distal esophageal, GE junction or gastric carcinoma, and that have progressed following treatment with front line fluoropyrimidine/platinum with or without trastuzumab is provided in which the patients are treated with paclitaxel+MM-111. The patients are treated with a regimen that follows a 4-week treatment cycle with dose administration of MM-111 once every 7±2 days. The anticancer therapies are administered by IV infusion in the following order:

a) Paclitaxel, which is administered for the first 3 weeks of the 4-week treatment cycle followed by 1 week off of paclitaxel therapy. Paclitaxel is preferably administered as an IV infusion over a period of about 60 minutes. The infusion should be prepared as directed in the paclitaxel package insert and in compliance with any institutional guidelines; and

b) MM-111, in which the first dose is administered over about 90 minutes followed by weekly dosing over about 60 minutes in the absence of infusion-related reactions.

In a preferred embodiment, the drugs are administered consecutively without any time interval between the administrations of each component of the regimen.

In another embodiment of any of the preceding aspects, the treatment produces at least one therapeutic effect selected from the group consisting of reduction in size of a tumor, reduction in number of metastatic lesions over time, complete response, partial response, stable disease, increase in overall response rate, and pathologic complete response.

In still another embodiment of any of the preceding aspects, the patient is additionally treated with G-CSF.

In a third aspect, a container is provided that comprises an effective amount of a bispecific anti-ErbB2/anti-ErbB3 antibody (e.g., an antibody comprising a polypeptide having an amino acid sequence as set forth in SEQ ID NO:1), and instructions for using the bispecific anti-ErbB2/anti-ErbB3 antibody according to the methods disclosed herein. In one embodiment, the container comprises at least 250 mg of the bispecific antibody (e.g., at least about 250 mg to about 1,000 mg).

In another embodiment, the container comprises an effective amount of one or more of lapatinib, docetaxel, paclitaxel, and trastuzumab.

### DETAILED DESCRIPTION

### I. Definitions

As used herein, the term “subject” or “patient” is a human cancer patient.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a marker of a cancer. Effective treatment may refer to alleviation of at least one symptom of a cancer. Such effective treatment may, e.g., reduce patient pain, reduce the size and/or number of lesions, may reduce or prevent metastasis of a cancer tumor, and/or may slow growth of a cancer tumor.

As used herein, “cancer” refers to a condition characterized by abnormal, unregulated, malignant cell growth. In some embodiments, the cancer tumor is a HER2+ solid tumor type, e.g., a melanoma, a cholangiocarcinoma, clear cell sarcoma, or an esophageal, head and neck, endometrial, prostate, breast, ovarian, gastric, gastro-esophageal junction (GEJ), colon, colorectal, lung, bladder, pancreatic, salivary gland, liver, skin, brain or renal tumor. In other embodiments, the cancer is squamous cell cancer, small-cell lung cancer, non-small cell lung cancer, cervical cancer, or thyroid cancer.

The terms “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to cancers, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay tumor development. In some embodiments, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit (i.e., slow to some extent and may stop) tumor metastasis; (v) inhibit tumor growth; (vi) prevent or delay occurrence and/or recurrence of tumor; and/or (vii) relieve to some extent one or more of the symptoms associated with the cancer. In one example, an effective amount for therapeutic uses is the amount of MM-111 and the amount of lapatinib and the amount of paclitaxel and the amount of trastuzumab clinically proven to effect as significant decrease in a cancer or slowing of progression of a cancer, such as an advanced solid tumor, e.g., that is HER-2 positive. In another example, an effective amount, an effective amount for therapeutic uses is the amount of MM-111 and the amount of docetaxel and the amount of trastuzumab clinically proven to effect as significant decrease in a cancer or slowing of progression of a cancer, such as an advanced solid tumor, e.g., that is HER-2 positive.

The term “antibody” includes antibodies and antibody variants comprising at least one antibody derived antigen binding site (e.g., VH/VL region or Fv) that specifically binds to ErbB2 or ErbB3. Antibodies include known forms of antibodies. For example, the antibody can be a human antibody, a humanized antibody, a bispecific antibody, or a chimeric antibody. The antibody also can be a Fab, Fab′2, ScFv, SMIP, Affibody®, nanobody, or a domain antibody. The antibody also can be of any of the following isotypes: IgG1, IgG2, IgG3, IgG4, IgM, IgA1, IgA2, IgAsec, IgD, and IgE.

As used herein, the term antibody variant includes naturally occurring antibodies which have been altered (e.g., by mutation, deletion, substitution, conjugation to a non-antibody moiety) to include at least one variant amino acid which changes a property of the antibody. For example, numerous such alterations are known in the art which affect, e.g., half-life, effector function, and/or immune responses to the antibody in a patient. The term antibody variant also includes artificial polypeptide constructs which comprise at least one antibody-derived binding site.

The term lapatinib (lapatinib ditosylate) refers to a dual tyrosine kinase inhibitor which disrupts the EGF and HER2 growth receptor pathways. It inhibits receptor signal processes by binding to the ATP-binding pocket of the EGFR/HER2 protein kinase domain, preventing phosphorylation and subsequent activation of the signal mechanism. Lapatinib is approved in combination with capecitabine, for the treatment of patients with advanced or metastatic breast cancer whose tumors overexpress HER2 and who have received prior therapy including an anthracycline, a taxane, and trastuzumab. It is also approved in combination with letrozole for the treatment of postmenopausal women with hormone receptor positive metastatic breast cancer that overexpresses the HER2 receptor for whom hormonal therapy is indicated. Lapatinib is marketed under the trade name TYKERB®.

Paclitaxel is a natural product with antitumor activity. The drug is produced via a semi-synthetic process from Taxus baccata. The chemical name for Paclitaxel is (5β,20-Epoxy-1,2α,4,7β,10β,13α-hexahydroxytax-11-en-9-one 4,10-diacetate 2-benzoate 13-ester with (2R,3S)-N-benzoyl-3-phenylisoserine. Paclitaxel is sold under the trade name TAXOL®. Albumin-bound paclitaxel, or nab-paclitaxel, is sold under the trade name ABRAXANE®.

The term docetaxel refers to the drug having the chemical name 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate}. Docetaxel is an antineoplastic agent belonging to the taxoid family. It is prepared by semisynthesis beginning with a precursor extracted from the renewable needle biomass of yew plants. Docetaxel is an antineoplastic agent that acts by disrupting the microtubular network in cells that is essential for mitotic and interphase cellular functions. Docetaxel is a mitotic inhibitor used in cancer chemotherapy to treat patients with lung cancer, ovarian cancer, breast cancer, head and neck cancer, and prostate cancer. Docetaxel stabilizes microtubules and as a result, interferes with the normal breakdown of microtubules during cell division. It is marketed under the trade name TAXOTERE®.

The term trastuzumab refers to a humanized monoclonal antibody that binds to a domain of the extracellular segment of the HER2 receptor. While its mechanism of action is not clear, cells treated with trastuzumab undergo arrest during the G1 phase of the cell cycle, reducing cell proliferation. It has been suggested that trastuzumab induces some of its effect by downregulation of HER2 leading to disruption of receptor dimerization and signaling through the downstream PI3K cascade. Also, trastuzumab suppresses angiogenesis by both induction of anti-angiogenic factors and repression of pro-angiogenic factors. Preclinical data also indicate that antibodies, including trastuzumab, when bound to a cell, induce antibody-dependent cell-mediated cytotoxicity. While trastuzumab treatment is now standard of care for HER2+ breast cancer, in vitro studies indicate that anti-HER2 monoclonal antibodies suppress the proliferation of ovarian, gastric, and NSCLC cell lines that overexpress the HER2 receptor. Therefore, anti-HER2 monoclonal antibodies may have important therapeutic significance in patients presenting with these or other human carcinomas. Trastuzumab is sold under the trade name HERCEPTIN®.

### II. Bispecific Anti-ErbB2/Anti-ErbB3 Antibodies

A number of bispecific anti-ErbB2/antiErbB3 antibodies that are scFv HSA conjugates are described in co-pending US patent publication No. 2011-0059076 and US patent publication No. 2012-003221 and in PCT publication Nos. WO2009/126920 and WO 2010/059315, each of which is incorporated herein by reference in its entirety and each of which discloses MM-111 (also referred to as B2B3-1) and other bispecific anti-ErbB2/antiErbB3 antibodies that are scFv HSA conjugates and that are suitable for use in the methods and compositions provided herein, including the components of A5-HSA-ML3.9, ML3.9-HSA-A5, A5-HSA-B1D2, B1D2-HSA-A5, B12-HSA-B1D2, B1D2-HSA-B12, A5-HSA-F5B6H2, F5B6H2-HSA-A5, H3-HSA-F5B6H2, F5B6H2-HSA-H3, F4-HSA-F5B6H2, F5B6H2-HSA-F4, B1D2-HSA-H3, and H3-HSA-B1D2. Other suitable bispecific anti-ErbB2/antiErbB3 antibodies are disclosed and claimed in U.S. Pat. Nos. 7,332,580 and 7,332,585, which are incorporated herein by reference.

A bispecific anti-ErbB2/anti-ErbB3 antibody (e.g., MM-111) can be co-administered with other therapeutic agents, (e.g, cisplatin, capecitabine, lapatinib, trastuzumab, docetaxel, paclitaxel, or nab-paclitaxel) prior to (e.g., neoadjuvant therapy), concurrent with, or following (e.g., adjuvant therapy) radiotherapy of, or surgical intervention to remove, a malignant tumor.

### III. Pharmaceutical Compositions

Pharmaceutical compositions suitable for administration to a patient are preferably in liquid form for intravenous administration.

In general, compositions typically comprise a pharmaceutically acceptable carrier. As used herein, the term “pharmaceutically acceptable” means approved by a government regulatory agency listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline and aqueous dextrose and glycerol solutions may be employed as carriers, particularly for injectable solutions (e.g., comprising a bispecific anti-ErbB2/anti-ErbB3 antibody and another therapeutic and one or more of lapatinib, paclitaxel, docetaxel, and/or trastuzumab). Liquid compositions for parenteral administration can be formulated for administration by injection or continuous infusion. Routes of administration by injection or infusion include intravenous, intraperitoneal, intramuscular, intrathecal and subcutaneous. In one embodiment, the anti-ErbB2/anti-ErbB3 antibody and either the combination of paclitaxel and trastuzumab or the anti-ErbB2/anti-ErbB3 antibody and the combination of docetaxel and trastuzumab are administered intravenously (e.g., separately or together over the course of one hour).

MM-111 may be prepared as a formulation containing 25 mg/ml MM-111 (e.g., about 1 mg/ml to about 100 mg/ml) in a sterile aqueous solution comprising 20 mM L-histidine hydrochloride, 150 mM sodium chloride, pH 6.5, which is stored at 2-8° C.

Preferably, MM-111 is brought to room temperature prior to administration and containers (e.g., vials) of MM-111 are not shaken. The appropriate quantity of MM-111 is removed from the container, diluted in 250 mL of 0.9% normal saline and administered as an infusion using a low protein binding in-line filter (preferably a 0.22 micrometer filter).

MM-111 is initially administered over about 90 minutes (first administration). In the absence of an infusion reaction, subsequent doses are administered over about 60 minutes.

A patient's body weight at the start of a dosing cycle is to be used to calculate the dose used throughout the cycle. Should a patient's body weight change by more than 10%, a new total dose is calculated to reflect this change.

Lapatinib dytosylate (TYKERB®, GSK) is an orally active drug for breast cancer and other solid tumors. It is available in 250 mg tablets and its bioavailability is increased with food consumption.

Lapatinib has the following structural formula:

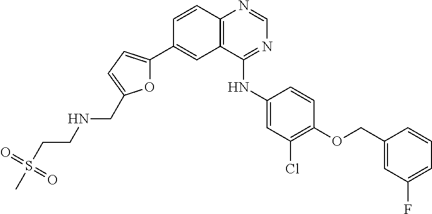 [SMILES: CS(=O)(=O)CCNCc1ccc(-c2ccc3ncnc(Nc4ccc(OCc5cccc(F)c5)c(Cl)c4)c3c2)o1]

Lapatinib has the chemical formula C₂₉H₂₆ClFN₄O₄S.

Paclitaxel injection, USP is a clear colorless to slightly yellow viscous solution. It is supplied as a non-aqueous solution intended for dilution with a suitable parenteral fluid prior to intravenous infusion. Paclitaxel is available in 30 mg (5 mL), 100 mg (16.7 mL), and 300 mg (50 mL) multidose vials. Each mL of sterile non-pyrogenic solution contains 6 mg Paclitaxel, 527 mg of polyoxyl 35 castor oil, NP and 49.7% (v/v) dehydrated alcohol, USP,

Paclitaxel has the following structural formula:

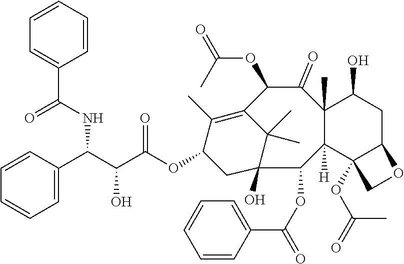 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Paclitaxel is a white to off-white crystalline powder with the molecular formula C₄₇H₅₁NO₁₄ and a molecular weight of 853.9, It is highly lipophilic, insoluble in water, and melts at around 216° C. to 217° C.

Docetaxel is the active ingredient available in 20 mg and 80 mg TAXOTERE® single-dose vials of concentrated anhydrous docetaxel in polysorbate 80. Docetaxel has the following structural formula:

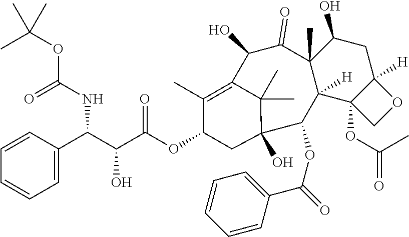

Docetaxel is a white powder with the molecular formula C₄₃H₅₃NO₁₄ and a molecular weight of 807.8. Docetaxel differs from paclitaxel at two positions in its chemical structure. It has a hydroxyl functional group on carbon 10, whereas paclitaxel has an acetate ester, and a tert-butyl carbamate, ester exists on the phenylpropionate side chain instead of the benzyl amide in paclitaxel. The carbon 10 functional group change causes docetaxel to be more water soluble than paclitaxel.

Hypersensitivity reaction may occur in patients treated with taxanes (e.g., fever, facial flushing, chills, shortness of breath, or hives), Anaphylaxis and severe hypersensitivity reactions characterized by dyspnea and hypotension requiring treatment, angioedema, and generalized urticaria have occurred in 2 to 4% of patients receiving paclitaxel in clinical trials. In some embodiments, a patient is given a pretreatment regimen with corticosteroids, diphenhydramine, and H2 antagonists.

Trastuzumab is a humanized monoclonal antibody targeting the ErbB2/HER2 receptor. Trastuzumab is approved for HER2-overexpressing breast cancer and HeR2-overexpressing metastatic gastric or gastro-esophageal junction adenocarcinoma. Trastuzumab is marketed under the trade name HERCEPTIN®.

### IV. Patient Populations

Provided herein are effective methods for treating patients with histologically or cytologically confirmed advanced cancer that is positive for HER2 (HER2+). HER2+ cancers are those in which the tumor cells overexpress HER2. A tumor that overexpresses HER2 is one that is identified as being HER2 “3+” or HER2 “2+” by immunohistochemistry (e.g., by HERCEPTEST®), or gene-amplified positive by fluorescence in situ hybridization (FISH+). In some embodiments, a tumor may be HER2+ as determined by immunohistochemistry but negative for HER2 as determined by FISH. Chromogenic in situ hybridization (CISH) may also be used if FISH results are unavailable. Patients can be tested or selected for one or more of the above described clinical attributes prior to, during or after treatment.

### V. Combination Therapy

As herein provided, bispecific anti-ErbB2/anti-ErbB3 antibodies are administered adjunctively with either the combination of lapatinib, paclitaxel, and trastuzumab or the combination of docetaxel and trastuzumab in combination with to effect improvement in subjects having HER2-positive solid tumors. In one embodiment, the bispecific anti-ErbB2/anti-ErbB3 antibody is an antibody having the amino acid sequence set forth in SEQ ID NO:1.

As used herein, adjunctive or combined administration (co-administration) includes simultaneous administration of the compounds in the same or different dosage form, or separate administration of the compounds (e.g., sequential administration). For example, the antibody can be simultaneously administered with paclitaxel, wherein both the antibody and paclitaxel are formulated together. Alternatively, the antibody can be administered in combination with one or more of lapatinib, paclitaxel, docetaxel, and trastuzumab, wherein both the antibody and the one or more other therapeutics are formulated for separate administration and are administered concurrently or sequentially. For example, lapatinib, paclitaxel, and trastuzumab can be administered prior to administration of the antibody, or vice versa. Such concurrent or sequential administration preferably results in both MM-111 and one or more of lapatinib, paclitaxel, docetaxel, and/or trastuzumab being simultaneously present in treated patients.

In another embodiment, bispecific anti-ErbB2/anti-ErbB3 antibody is formulated for intravenous administration. In particular embodiments, the bispecific anti-ErbB2/anti-ErbB3 antibody is administered at a dose selected from: of 40 mg/kg, 30 mg/kg, 20 mg/kg, 15 mg/kg, 12 mg/kg, 10 mg/kg, and/or 5 mg/kg. In one embodiment, the dose of antibody is varied over time. For example, the antibody may be initially administered at a high dose and may be lowered over time, e.g., a 40 mg/kg dose may be lowered to a 35 mg/kg dose, or a 20 mg kg may be lowered to a 15 mg/kg dose. In another embodiment, the antibody is initially administered at a low dose and increased over time.

### VI. Treatment Protocols

Suitable treatment protocols include, for example, those wherein a patient (i.e., human subject) receives a daily dose of (A) lapatinib (about 750 or about 1000 mg by mouth (PO) within an hour of ingesting food); a weekly dose of (B) paclitaxel (by IV infusion over 60 minutes) at a dose of about 80 mg/m²; a weekly dose of (C) trastuzumab (by IC infusion of 90 minutes) at a loading dose of 4 mg/kg for the first week followed by a maintenance dose of 2 mg/kg thereafter; and a weekly dose of (D) the bispecific anti-ErbB2/anti-ErbB3 antibody (by IV infusion over 90 minutes the first week and over 60 minutes thereafter) at a starting dose of about 20 mg/kg. Another exemplary treatment protocol is one wherein a patient receives a dose every three weeks of (A) docetaxel (by IV infusion over 60 minutes) at a dose of about 75 mg/m²; (B) trastuzumab (at a loading dose of about 8 mg/kg for the first IV infusion over 90 minutes, followed by infusions of about 6 mg/kg over 60 minutes thereafter); and (C) a bispecific anti-ErbB2/anti-ErbB3 antibody (at a starting dose of about 30 mg/kg by IV infusion over 90 minutes the first week and over 60 minutes thereafter).

In another embodiment, the amount of the bispecific anti-ErbB2/anti-ErbB3 antibody administered is constant for each dose. In another embodiment, the amount of antibody administered varies with each dose. For example, the maintenance (or follow-on) dose of the antibody can be higher or the same as the loading dose which is first administered. In another embodiment, the maintenance dose of the antibody can be lower or the same as the loading dose. In one embodiment, a bispecific anti-ErbB2/anti-ErbB3 antibody is administered as a monotherapy prior to at least one cycle of bispecific anti-ErbB2/anti-ErbB3 antibody combination therapy.

### VII. Outcomes

Responses to therapy may include:

Pathologic complete response (pCR): absence of invasive cancer in the breast and lymph nodes following primary systemic treatment.
Complete Response (CR): Disappearance of all target lesions. Any pathological lymph nodes (whether target or non-target) which has reduction in short axis to <10 mm;
Partial Response (PR): At least a 30% decrease in the sum of dimensions of target lesions, taking as reference the baseline sum diameters;
Stable Disease (SD): Neither sufficient shrinkage to qualify for partial response, nor sufficient increase to qualify for progressive disease, taking as reference the smallest sum diameters while on study; or

Meanwhile, non-CR/Non-PD denotes a persistence of one or more non-target lesion(s) and/or maintenance of tumor marker level above the normal limits.

Progressive Disease (PD) denotes at least a 20% increase in the sum of dimensions of target lesions, taking as reference the smallest sum on study (this includes the baseline sum if that is the smallest on study). In addition to the relative increase of 20%, the sum must also demonstrate an absolute increase of 5 mm. The appearance of one or more new lesions is also considered progression;

In exemplary outcomes, patients treated according to the methods disclosed herein may experience improvement in at least one sign of breast cancer.

In one embodiment the patient so treated exhibits pCR, CR, PR, or SD.

In another embodiment, the patient so treated experiences tumor shrinkage and/or decrease in growth rate, i.e., suppression of tumor growth. In another embodiment, unwanted cell proliferation is reduced or inhibited. In yet another embodiment, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

In other embodiments, such improvement is measured by a reduction in the quantity and/or size of measurable tumor lesions. Measurable lesions are defined as those that can be accurately measured in at least one dimension (longest diameter is to be recorded) as ¢10 mm by CT scan (CT scan slice thickness no greater than 5 mm), 10 mm caliper measurement by clinical exam or >20 mm by chest X-ray. The size of non-target lesions, e.g., pathological lymph nodes can also be measured for improvement. In one embodiment, lesions can be measured on chest x-rays or CT or MRI films.

In other embodiments, cytology or histology can be used to evaluate responsiveness to a therapy. The cytological confirmation of the neoplastic origin of any effusion that appears or worsens during treatment when the measurable tumor has met criteria for response or stable disease can be considered to differentiate between response or stable disease (an effusion may be a side effect of the treatment) and progressive disease.

In some embodiments, administration of effective amounts of the bispecific anti-ErbB2/anti-ErbB3 antibody and either the combination of lapatinib, paclitaxel and trastuzumab, or the combination of docetaxel and trastuzumab according to any of the methods provided herein produce at least one therapeutic effect selected from the group consisting of reduction in size of a breast tumor, reduction in number of metastatic lesions appearing over time, complete remission, partial remission, stable disease, increase in overall response rate, or a pathologic complete response. In some embodiments, the provided methods of treatment produce a comparable clinical benefit rate (CBR=CR+PR+SD≧6 months) better than that achieved by either the combination of lapatinib, paclitaxel and trastuzumab, or by the combination of docetaxel and trastuzumab. In other embodiments, the improvement of clinical benefit rate is about 20%, 30%, 40%, 50%, 60%, 70%, 80% or more as compared to either the combination of lapatinib, paclitaxel and trastuzumab or the combination of docetaxel and trastuzumab in the absence of MM-111.

### VIII. Kits and Unit Dosage Forms

Also provided are kits that include a pharmaceutical composition containing a bispecific anti-ErbB2/anti-ErbB3 antibody, such as MM-111, and a pharmaceutically-acceptable carrier, in a therapeutically effective amount adapted for use in the preceding methods. The kits can optionally also include instructions, e.g., comprising administration schedules, to allow a practitioner (e.g., a physician, nurse, or patient) to administer the composition contained therein to administer the composition to a patient having a cancer. In one embodiment, the kit further comprises one or more of paclitaxel, lapatinib, docetaxel, and trastuzumab. In one embodiment, the kit includes MM-111 in sterile, single-use vials containing 10.1 mL of MM-111 at a concentration of 25 mg/ml in 20 mM histidine, 150 mM sodium chloride, pH 6.5. In another embodiment the kit includes a syringe. In another embodiment, the kit includes a low protein binding 0.22 micrometer in-line filter.

Optionally, the kits include multiple packages of the single-dose pharmaceutical composition(s) containing an effective amount of the antibody (e.g., MM-111) for a single administration in accordance with the methods provided above. Optionally, instruments or devices necessary for administering the pharmaceutical composition(s) may be included in the kits. For instance, a kit may provide one or more pre-filled syringes containing an amount of MM-111 that is about 100 times the dose in mg/kg indicated for administration in the above methods. Optionally, the kit may further comprise one or more of paclitaxel, lapatinib, docetaxel, and/or trastuzumab in a desired unit dosage form (e.g., a unit dosage form distributed by the manufacturer of paclitaxel, lapatinib, docetaxel, and/or trastuzumab) for administration.

The following examples are merely illustrative and should not be construed as limiting the scope of this disclosure in any way as many variations and equivalents will become apparent to those skilled in the art upon reading the present disclosure.

### EXAMPLES

### Clinical Trials in Humans: Study Design

In one embodiment, a human clinical trial study is an open-label, multicenter, dose-escalation study of MM-111 in an add-on design in combination with one of the following treatments: cisplatin, capecitabine, and trastuzumab, lapatinib and trastuzumab, paclitaxel and trastuzumab; lapatinib, paclitaxel and trastuzumab; or docetaxel and trastuzumab. MM-111 and the combination treatments will be administered in cycles as described in the Examples below. The safety assessment period for purposes of dose limiting toxicity (DLT) evaluation and dose escalation decisions will be one complete cycle (21 days for a 3-week cycle and 28 days for a 4-week cycle).

Patients with any HER2+ solid tumor type who have failed previous standard therapy may be enrolled. This study is a standard 3+3 design. For the regimen described in Example 1 below (lapatinib, trastuzumab and paclitaxel) the starting dose is 20 mg/kg for MM-111 and MM-111 will be administered once per week. For the regimen described in Example 2 below (docetaxel and trastuzumab) the MM-111 starting dose is 30 mg/kg and MM-111 will be administered every three weeks. If a DLT is observed in 1 of 3 patients during a cycle, the cohort will be expanded to 6 patients. If a second DLT is observed at that dose, then the previous dose level will be determined to be the maximum tolerable dose (MTD); however, intermediate dose levels may be evaluated. If there is not a second DLT, then dosing will proceed to the next dose level of MM-111 and the combination regimen up to the highest dose level specified for each combination therapy. If patients experience excessive toxicity at the highest dose level, they can receive a lower dose of MM-111 (e.g., 20→15 mg/kg). Blood will be drawn as noted in the schedule of assessments before and after the first administration of the agents together to determine the PK of MM-111 in combination with the other treatments.

### Example 1

### Treatment with Lapatinib, Paclitaxel, Trastuzumab and MM-111

Lapatinib and trastuzumab have been shown to have demonstrated synergy when compared to the individual agents (Blackwell et al., 2010). The combination of trastuzumab and paclitaxel is an effective regimen in HER2-positive breast cancer patients. Recently, Baselga et al. reported the results of a neoadjuvant study in which patients were randomized into three arms to receive either (1) lapatinib alone 1500 mg orally daily (N=154) or (2) trastuzumab loading dose 4 mg/kg and 2 mg/kg maintenance (N=149) or (3) the combination of lapatinib (1000 mg) and trastuzumab (N=152), for six weeks. After that, patients received the same treatments in combination with paclitaxel 80 mg/m² weekly for another twelve weeks. The primary endpoint of this phase III study (NeoALTTO) was pathologic complete response rate (pCR).

The pCR rate was significantly higher in the group given lapatinib and trastuzumab (78 of 152 patients [51·3%; 95% CI 43·1-59·5]) than in the group given trastuzumab alone (44 of 149 patients [29·5%; 22·4-37·5]; difference 21·1%, 9·1-34·2, p=0·0001). There were no significant difference in pCR between the lapatinib (38 of 154 patients [24·7%, 18·1-32·3]) and the trastuzumab (difference −4·8%, −17·6 to 8·2, p=0·34) groups. No major cardiac dysfunctions occurred. Frequency of grade 3 diarrhea was higher with lapatinib (36 patients [23·4%]) and lapatinib plus trastuzumab (32 [21·1%]) than with trastuzumab alone (three [2·0%]). For the trastuzumab lapatinib arm, this lead to a protocol amendment that decreased the dose of lapatinib to 750 mg. Similarly, grade 3 liver-enzyme alterations were more frequent with lapatinib (27 [17·5%]) and lapatinib plus trastuzumab (15 [9·9%]) than with trastuzumab (11 [7·4%]).

To summarize, the lapatinib, trastuzumab and paclitaxel regimen is effective and reasonably well tolerated. Pre-clinically MM-111 is additive to all three drugs (lapatinib, trastuzumab, paclitaxel) both as individual agents and in combinations. To date, there has not been any evidence of overlapping toxicity with these combinations so therefore (and substantiated by the NeoALTTO data above) there is a strong interest in evaluating the safety and efficacy of the four drug combination.

### Treatment Regimen: Lapatinib, Paclitaxel, and Trastuzumab+MM 111

The regimen for lapatinib+trastuzumab+paclitaxel+MM-111 follows a 4-week treatment cycle. The anticancer therapies will be administered in the following order: 1) Lapatinib 2) Paclitaxel 3) Trastuzumab and 4) MM-111.

| | Lapatinib | Paclitaxel | Trastuzumab | MM-111 |
|---|---|---|---|---|
| Level | (mg) ^{a} | (mg/m²) ^{b} | (mg/kg)^{c} | (mg/kg)^{d} |
| −2 | 750 | 80 | 2 | 5 |
| −1 | 750 | 80 | 2 | 10 |
| 1 | 750 | 80 | 2 | 20 |
| 2^{e} | 1000 | 80 | 2 | 20 |

^{a} 250 mg tablets taken orally daily within an hour of ingesting food taken, and on days of infusion it is to be taken just before infusions are given.
^{b} Paclitaxel is administered at 80 mg/m² as an IV infusion weekly over 60 minutes. The infusion is prepared as directed in the paclitaxel package insert and any institutional guidelines. All patients receiving paclitaxel are pre-medicated as per the local institutional guidelines.
^{c}The first dose of trastuzumab is a loading dose of 4 mg/kg administered over 90 minutes followed by weekly dosing at 2 mg/kg over 30 minutes via IV infusion.
^{d}The first dose of MM-111 is administered over 90 minutes followed by weekly dosing over 60 minutes in the absence of infusion-related reactions.
^{e}Level 2 may be enrolled based on an evaluation of the safety and PK data from proceeding dose levels.

a 250 mg tablets taken orally daily within an hour of ingesting food taken, and on days of infusion it is to be taken just before infusions are given.

b Paclitaxel is administered at 80 mg/m² as an IV infusion weekly over 60 minutes. The infusion is prepared as directed in the paclitaxel package insert and any institutional guidelines. All patients receiving paclitaxel are pre-medicated as per the local institutional guidelines.

c The first dose of trastuzumab is a loading dose of 4 mg/kg administered over 90 minutes followed by weekly dosing at 2 mg/kg over 30 minutes via IV infusion.

d The first dose of MM-111 is administered over 90 minutes followed by weekly dosing over 60 minutes in the absence of infusion-related reactions.

e Level 2 may be enrolled based on an evaluation of the safety and PK data from proceeding dose levels.

Paclitaxel dosing should begin first dose on Cycle 1 Day 1. The infusion should be prepared as directed in the paclitaxel package insert. All patients receiving paclitaxel should be pre-medicated as per the local institutional guidelines. In addition, for treatment details and dose modifications, sites should also refer to their institutional guidelines.

Treatment with this regimen will be continued until disease progression, unacceptable toxicity, or withdrawal of consent. However, if a toxicity is isolated to one drug within the combination, (for example, neuropathy develops due to paclitaxel), treatment may continue with the remaining agents until progression.

The following adverse events (AEs) are relatively common and to be expected with the combination of lapatinib and trastuzumab. Related Grade 3 events with the combination include diarrhea (17%), fatigue (11%), and rash (6%). The following AEs are relatively common and to be expected with the combination of lapatinib, trastuzumab and paclitaxel: infusion reactions and hematologic toxicities.

For this combination, the following adverse events will be considered as DLTs when occurring during Cycle 1, if the relatedness criterion is at least ‘probable’ or ‘definite’ or ‘unknown’ and if not related to disease progression.

Grade 4 neutropenia >7 days or Grade 3 or 4 neutropenia complicated by fever ≧38.5° C. (i.e., febrile neutropenia) and/or documented infection

Grade 3 or 4 thrombocytopenia and/or anemia >7 days or any Grade 3 or 4 thrombocytopenia complicated with hemorrhage;

Grade 3 or 4 non-hematologic toxicity, (except fatigue/asthenia <2 weeks in duration, anorexia, nausea/vomiting in the absence of optimal anti-emetics, diarrhea in the absence of optimal anti-diarrheals, alkaline phosphatase changes, or alopecia).

Grade 3 or 4 infusion reactions related to MM-111

Grade 3 or 4 rash lasting longer than 2 weeks

Inability to deliver all 4 of the planned MM-111 doses over the first cycle of treatment due to drug-related toxicities

Grade 3 or 4 infusion reactions directly attributable to MM-111 administration The following will not be considered dose limiting:

Grade 3 or 4 infusion reactions due to paclitaxel administration

Grade 3 elevation in transaminases, total bilirubin, or alkaline phosphatase levels

Lapatinib and trastuzumab have also been associated with cardiac dysfunction. A DLT for cardiac dysfunction will include any heart failure that is >Grade 2 or greater by NCI CTCAE version 4.0 or any in patients with a LVEF that drops below the institution's LLN.

Patients must have adequate hepatic function as evidenced by 1) serum total bilirubin ≦1.5× the upper limit of normal (ULN) and 2) aspartate aminotransferase (AST), alanine aminotransferase (ALT), and alkaline phosphatase ≦2.5×ULN (5×ULN is acceptable if liver metastases are present).

Any other toxicities that are clearly related to the regimen and unexpected of MM-111 will be discussed between the Investigator, Medical monitor and Sponsor before being assigned the category of DLT in Cycle 1. If there is evidence that a patient who experiences a DLT has also derived clinical benefit from treatment with MM-111, then the Investigators, Medical Monitor, and Sponsor will review the specifics of the case. Such a patient may continue on study at the next lower dose level if the consensus judgment is that continued treatment is in the patient's best interest.

### Example 2

### Treatment with Docetaxel and Trastuzumab+MM-111

Pre-clinically, the combination of docetaxel and MM-111 is additive from an efficacy standpoint. By inhibition of ErbB3, the addition of MM-111 to such a regimen may prevent resistance to HER directed therapy and tumor regrowth and hypothetically could potentiate the efficacy of the effective regimen.

In the current multi-arm study, MM-111 has been combined with a taxane (paclitaxel) and trastuzumab given weekly and has been well tolerated to date. There is no evidence of any overlapping toxicities of paclitaxel, trastuzumab and MM-111. Both the three week docetaxel regimen in combination with trastuzumab and weekly paclitaxel in combination with trastuzumab are approved for HER2 positive breast cancer (FDA; HERCEPTIN® [trastuzumab] U.S. Package Insert 2010). It is ultimately the intent to develop an every three week regimen comprising of docetaxel, trastuzumab and MM-111. Such a regimen would be useful in evaluating the role of MM-111 when added to standard (every three week) combinations of taxane and trastuzumab that are used in HER2-positive disease.

The combination of docetaxel, trastuzumab and pertuzumab is undergoing review for possible approval following the report of the CLEOPATRA study (Baselga, New England Journal 2011). In that study this combination improved progression free survival by six months in the front line setting in patients with metastatic HER2 positive breast cancer, when compared to docetaxel and trastuzumab alone. This triplet regimen has also been evaluated in the neoadjuvant setting and compared to docetaxel and trastuzumab in the Neosphere study (Gianni et al, Lancet 2012). Patients treated with the triplet (pertuzumab and trastuzumab plus docetaxel) had a significantly improved pCR (45·8% [95% CI 36·1-55·7]) compared with those given trastuzumab plus docetaxel (29·0% [20·6-38·5]; p=0·0141). (24·0% [15·8-33·7]) women given pertuzumab plus docetaxel had a pCR, as did (16·8% [10·3-25·3]) given pertuzumab and trastuzumab. It is possible that this will become a new standard of care of HER2 positive breast cancer patients if approved. Once every three week regimen of MM-111 would be useful to benchmark against the pertuzumab based regiment described in the CLEOPATRA study above.

### Treatment Regimen: Docetaxel, Trastuzumab+MM-111

The regimen for treatment with docetaxel and trastuzumab and MM-111 will follow a 3-week treatment cycle. The anti-cancer therapies will be administered in the following order: 1) Docetaxel, 2) Trastuzumab, and 3) MM-111.

| | Docetaxel | Trastuzumab | MM-111 |
|---|---|---|---|
| Level | (mg/m²) ^{a} | (mg/kg)^{b} | (mg/kg)^{c} |
| −2 | 75 | 6 | 15 |
| −1 | 75 | 6 | 20 |
| 1 | 75 | 6 | 30 |
| 2 | 75 | 6 | 40 |

^{a} Docetaxel dosing should begin first dose on Cycle 1, Day 1, and is administered as an IV infusion over 60 minutes every three weeks. The infusion should be prepared as directed in the docetaxel package insert and any institutional guidelines. All patients receiving docetaxel should be pre-medicated as per the local institutional guidelines.
^{b}The first dose of trastuzumab is a loading dose of 8 mg/kg administered over 90 minutes followed by every three week dosing at 6 mg/kg over 60 minutes via IV infusion.
^{c}The first dose of MM-111 is administered over 90 minutes followed by 3 week dosing over 60 minutes in the absence of infusion-related reactions.

Up to six 3-week cycles of docetaxel will be administered. Beyond that, it is up to the PIs discretion to continue treatment with docetaxel until disease progression, unacceptable toxicity or withdrawal of consent. Treatment with MM-111 and trastuzumab will be continued until disease progression, unacceptable toxicity or withdrawal of consent.

Prophylactic use of G-CSF will be permitted only in those patients who have had at least one episode of grade 3 or 4 neutropenia or neutropenic fever while receiving study therapy.

Infusion reactions, fluid retention and hematologic toxicities are common with docetaxel.

For this combination, the following adverse events will be considered as DLTs when occurring during Cycle 1, if the related-ness criterion is at least ‘probable’ or ‘definite’ or ‘unknown’ and if not related to disease progression.

Grade 3 or 4 thrombocytopenia and/or anemia >7 days or any Grade 3 or 4 thrombocytopenia complicated with hemorrhage;

Grade 4 neutropenia >7 days or Grade 3 or 4 neutropenia complicated by fever ≧38.5° C. (i.e., febrile neutropenia) and/or documented severe infection

Any Grade 3 or 4 non-hematologic toxicity (except fatigue/asthenia <2 weeks in duration, anorexia, nausea/vomiting in the absence of optimal anti-emetics, diarrhea in the absence of optimal anti-diarrheals, alkaline phosphatase changes or alopecia).

Grade 3 or 4 infusion reactions directly attributable to MM-111 administration

A DLT for cardiac dysfunction will include any heart failure that is >Grade 2 NCI CTCAE (version 4.0) or any in patients with an LVEF that drops below the institution's LLN.

The following will not be considered dose limiting:

Grade 3 or 4 infusion reactions due to docetaxel administration

Grade 3 elevation in transaminases, total bilirubin, or alkaline phosphatase levels

Patients must have adequate hepatic function as evidenced by 1) serum bilirubin within normal limits, and AST and/or ALT<1.5×ULN and alkaline phosphatase <2.5×ULN if concomitantly elevated.

Any other toxicities will be discussed between the investigator, medical monitor and sponsor before being assigned the category of DLT in Cycle 1. If there is evidence that a patient who experiences a DLT has also derived clinical benefit from treatment with MM-111, then the Investigators, Medical Monitor, and Sponsor will review the specifics of the case. Such a patient may continue on study at the next lower dose level if the consensus judgment is that continued treatment is in the patient's best interest.

For patients receiving docetaxel and trastuzumab, use of granulocyte colony-stimulating factors (G-CSF) is permitted to treat patients with neutropenia or neutropenic fever, prophylactic use of G-CSF will be permitted only in those patients who have a history of at least one episode of grade 3 or 4 neutropenia or neutropenic fever with previous cytotoxic therapy.

### Example 3

### Treatment with Capecitabine, Cisplatin, and Trastuzumab+MM-111

Patients with HER2-positive cancer (e.g., HER2 2+ or HER2 3+) are treated with MM-111 combination therapy. In one embodiment, patients with previously untreated HER2+ metastatic gastric or GEJ cancers can be enrolled onto the cisplatin, capecitabine, and trastuzumab+MM-111 arm of the study. This study is a standard 3+3 design. The one embodiment, the initial dose of MM-111 is 10 mg/kg. In some embodiments, the initial dose of MM-111 is 5 mg/ml, although in other embodiments, MM-111 can be administered at an initial dose of, e.g., from about 500 μg/mL to about 5.0 mg/mL. In some embodiments, the dose of capecitabine is reduced from 1000 mg/m² to 800 mg/m².

The anticancer therapies should be administered in the following order: 1) Capecitabine, 2) Cisplatin, 3) Trastuzumab, and 4) MM-111

| | Cisplatin | Capecitabine | Trastuzumab | MM-111 |
|---|---|---|---|---|
| Level | (mg/m²)^{a} | (mg/m²)^{b} | (mg/kg)^{c} | (mg/kg)^{d} |
| −1 | 80 | 1000 | 6 | 5 |
| 1 | 80 | 1000 | 6 | 10 |
| 2 | 80 | 1000 | 6 | 20 |

^{a}Given on Day 1 every three weeks for six cycles via IV infusion over 2 hours. All patients receiving cisplatin should be pre-medicated as per the package insert and any local institutional guidelines.
^{b}Administered orally twice daily at consistent times of day for 14 days of a 21-day (3 week) cycle.
^{c}The first dose of trastuzumab is a loading dose of 8 mg/kg administered over 90 minutes followed by dosing at 6 mg/kg over 30-90 minutes via IV infusion every three weeks.
^{d}The first dose of MM-111 is administered over 90 minutes followed by weekly dosing over 60 minutes in the absence of infusion-related reactions.

### Example 4

### Treatment with Lapatinib+/−Trastuzumab+MM-111

This regimen follows a 4-week treatment cycle. The anticancer therapies should be administered in the following order:

### 1) Lapatinib, 2) Trastuzumab, and 3) MM-111.

| | Lapatinib | Trastuzumab | MM-111 |
|---|---|---|---|
| Level | (mg) ^{a} | (mg/kg)^{b} | (mg/kg)^{c} |
| −1 | 1000 | 2 | 5 |
| 1 | 1000 | 2 | 10 |
| 2 | 1000 | 2 | 20 |
| 3^{d} | 1250 | 0 | 20 |
| 4^{d, e} | 1500 | 0 | 20 |

^{a} 250 mg tablets taken orally daily within an hour of ingesting food in clinic during infusion days.
^{b}The first dose of trastuzumab is a loading dose of 4 mg/kg administered over 90 minutes followed by weekly dosing at 2 mg/kg over 30 minutes via IV infusion.
^{c}The first dose of MM-111 is administered over 90 minutes followed by weekly dosing over 60 minutes in the absence of infusion-related reactions
^{d}Patients who are hormone receptor positive may be given letrozole 2.5 mg orally daily at the PI discretion
^{e}Level 4 may be enrolled based, on an evaluation to the safety and PK data from levels −1-4

### Example 5

### Amino Acid Sequence of MM-111(SEQ ID NO:1)

QVQLQESGGGLVKPGGSLRLSCAASGFTFSSYWMSWVRQAPGKGLEWVAN
INRDGSASYYVDSVKGRFTISRDDAKNSLYLQMNSLRAEDTAVYYCARDR
GVGYFDLWGRGTLVTVSSASTGGGGSGGGGSGGGGSQSALTQPASVSGSP
GQSITISCTGTSSDVGGYNFVSWYQQHPGKAPKLMIYDVSDRPSGVSDRF
SGSKSGNTASLIISGLQADDEADYYCSSYGSSSTHVIFGGGTKVTVLGAA
SDAHKSEVAHRFKDLGEENFKALVLIAFAQYLQQSPFEDHVKLVNEVTEF
AKTCVADESAENCDKSLHTLFGDKLCTVATLRETYGEMADCCAKQEPERN
ECFLQHKDDNPNLPRLVRPEVDVMCTAFHDNEETFLKKYLYEIARRHPYF
YAPELLFFAKRYKAAFTECCQAADKAACLLPKLDELRDEGKASSAKQRLK
CASLQKFGERAFKAWAVARLSQRFPKAEFAEVSKLVTDLTKVHTECCHGD
LLECADDRADLAKYICENQDSISSKLKECCEKPLLEKSHCIAEVENDEMP
ADLPSLAADFVESKDVCKNYAEAKDVFLGMFLYEYARRHPDYSVVLLLRL
AKTYETTLEKCCAAADPHECYAKVFDEFKPLVEEPQNLIKQNCELFEQLG
EYKFQNALLVRYTKKVPQVSTPTLVEVSRNLGKVGSKCCKHPEAKRMPCA
EDYLSVVLNQLCVLHEKTPVSDRVTKCCTESLVNRRPCFSALEVDETYVP
KEFQAETFTFHADICTLSEKERQIKKQTALVELVKHKPKATKEQLKAVMD
DFAAFVEKCCKADDKETCFAEEGKKLVAASQAALGLAAALQVQLVQSGAE
VKKPGESLKISCKGSGYSFTSYWIAWVRQMPGKGLEYMGLIYPGDSDTKY
SPSFQGQVTISVDKSVSTAYLQWSSLKPSDSAVYFCARHDVGYCTDRTCA
KWPEWLGVWGQGTLVTVSSGGGGSSGGGSGGGGSQSVLTQPPSVSAAPGQ
KVTISCSGSSSNIGNNYVSWYQQLPGTAPKLLIYDHTNRPAGVPDRFSGS
KSGTSASLAISGFRSEDEADYYCASWDYTLSGWVFGGGTKLTVLG

### Example 6

### Paclitaxel+Trastuzumab+MM-111

Patients with HER2-positive cancer (e.g., HER2 2+ or HER2 3+) are treated with MM-111 combination therapy. In one embodiment, patients are treated who have metastatic or locally advanced (unresectable) HER2-expressing distal esophageal, GE junction or gastric carcinoma, and have progressed following treatment with front line fluoropyrimidine/platinum with or without trastuzumab. In one embodiment, the patients are HER2 2+ or HER2 3+ by IHC. In some embodiments, the patients are HER2 2+ and FISH positive. The patients are treated with a regimen that follows a 4-week treatment cycle with dose administration of MM-111 and trastuzumab once every 7±2 days. The anticancer therapies will be administered by IV infusion in the following order: 1) Paclitaxel, 2) Trastuzumab, and 3) MM-111. Study drugs should be administered consecutively without any time interval between the administrations of each component of the regimen. Paclitaxel should be administered for the first 3 weeks of the 4-week treatment cycle followed by 1 week off of paclitaxel therapy.

Paclitaxel is administered as an IV infusion over a period of about 60 minutes. The infusion is prepared as directed in the paclitaxel package insert and in compliance with any institutional guidelines. All patients receiving paclitaxel and trastuzumab should be pre-medicated as per the local institutional guidelines.

The first dose of trastuzumab is a loading dose of about 4 mg/kg administered over a period of about 90 minutes followed by weekly dosing at about 2 mg/kg over about 30 minutes by IV infusion.

The first dose of MM-111 is administered over a period of about 90 minutes followed by weekly dosing over about 60 minutes in the absence of infusion-related reactions.

| | Paclitaxel | Trastuzumab | MM-111 |
|---|---|---|---|
| Level | (mg/m²) ^{a} | (mg/kg)^{b} | (mg/kg)^{c} |
| −1 | 80 | 2 | 5 |
| 1 | 80 | 2 | 10 |
| 2 | 80 | 2 | 20 |

^{a} Paclitaxel is administered as an IV infusion over 60 minutes. The infusion should be prepared as directed in the paclitaxel package insert and any institutional guidelines. All patients receiving paclitaxel should be premedicated as per the local institutional guidelines.
^{b}The first dose of trastuzumab is a loading dose of 4 mg/kg administered over 90 minutes followed by weekly dosing at 2 mg/kg over 60 minutes via IV infusion.
^{c}The first dose of MM-111 is administered over 90 minutes followed by weekly dosing over 60 minutes in the absence of infusion-related reactions

### Example 7

### Paclitaxel+MM-111

Patients with HER2-positive cancer (e.g., HER2 2+ or HER2 3+) are treated with MM-111 combination therapy. In one embodiment, patients are treated who have metastatic or locally advanced (unresectable) HER2-expressing distal esophageal, GE junction or gastric carcinoma, and have progressed following treatment with front line fluoropyrimidine/platinum with or without trastuzumab. In one embodiment, the patients are HER2 2+ or HER2 3+ by IHC. In some embodiments, the patients are HER2 2+ and FISH negative. These patients are treated with a regimen that follows a 4-week treatment cycle with dose administration of MM-111 once every 7±2 days. The anticancer therapies are administered by IV infusion in the following order: 1) Paclitaxel and 2) MM-111. Study drugs are administered consecutively without any time interval between the administrations of each component of the regimen.

Paclitaxel is administered for the first 3 weeks of the 4-week treatment cycle followed by 1 week off of paclitaxel therapy. Paclitaxel is administered as an IV infusion over a period of about 60 minutes. The infusion should be prepared as directed in the paclitaxel package insert and in compliance with any institutional guidelines. All patients receiving paclitaxel should be pre-medicated as per the local institutional guidelines.

The first dose of MM-111 is administered over about 90 minutes followed by weekly dosing over about 60 minutes in the absence of infusion-related reactions.

### Dosing Scheme

| | Paclitaxel | MM-111 |
|---|---|---|
| | (mg/m²) | (mg/kg) |
| Dose | 80 | 20 |

Paclitaxel is administered as an IV infusion over a period of 60 minutes. The infusion is prepared as directed in the paclitaxel package insert and in compliance with any institutional guidelines. All patients receiving paclitaxel should be pre-medicated as per the local institutional guidelines.

The first dose of MM-111 is administered over 90 minutes followed by weekly dosing over 60 minutes in the absence of infusion-related reactions.

### Example 8

### Maintenance Dosing

In another embodiment, following the treatment regimens of any of the preceding Examples, patients who have received at least 6 cycles of chemotherapy and have stable disease or better will be administered maintenance therapy. Patients will proceed with maintenance treatment within 28 days from the date of completion of the chemotherapy. HER2 IHC-positive patients will be given a maintenance dose of trastuzumab alone or the combination of trastuzumab and a bispecific anti-HER2, anti-HER3 antibody.

Those skilled in the art will recognize, and is able to ascertain and implement using no more than routine experimentation, many equivalents of the specific embodiments described herein. Such equivalents are intended to be encompassed by the following claims. Any combinations of the embodiments disclosed in the dependent claims are within the scope of the disclosure.

All patents, patent applications and publications cited herein are incorporated herein by reference in their entireties.

1. A method for treatment of a cancer in a human patient, the method comprising administration to the patient of an effective amount of (a) a bispecific anti-ErbB2/anti-ErbB3 antibody, and one or more of
(b) lapatinib,
(c) a taxane that is paclitaxel, and
(d) trastuzumab, wherein the treatment comprises a first cycle of administration and at least one subsequent cycle of administration, wherein each cycle of administration spans a period of four weeks, and wherein:
during each of the four weeks of the first cycle,
(a) is administered at a weekly dose of at least 5 mg/kg,
(b) is administered at a daily dose of at least 750 mg,
(c) is administered at a weekly dose of at least 80 mg/m², and
(d) is administered at a weekly dose of at least 2 mg/kg, and during each of the four weeks of each subsequent cycle,
(a) is administered at a weekly dose of about 5, about 10, about 20, about 30, or about 50 mg/kg.
(b) is administered at a daily dose of about 750 or about 1000 mg,
(c) is administered at a weekly dose of about 80 mg/m², and
(d) is administered at a weekly dose of about 2 mg/kg.

2. The method of claim 1, wherein at least initial dosing during the first cycle of administration, one or more of (a), (b), (c) and (d) is at a loading dose that is greater than corresponding doses of one or more of (a), (b), (c) and (d) administered in each subsequent cycle.

3. A method for treatment of a cancer in a human patient, the method comprising administration to the patient of an effective amount of (a) a bispecific anti-ErbB2/anti-ErbB3 antibody, (e) a taxane that is docetaxel and (d) trastuzumab, wherein the treatment comprises a first cycle of administration and at least one subsequent cycle of administration, wherein each cycle is a period of three weeks, and wherein:
once during the first cycle:
(a) is administered at a dose of at least 15 mg/kg,
(d) is administered at a dose of at least 6 mg/kg, and
(e) is administered at a dose of at least 75 mg/m², and once during each subsequent cycle:
(a) is administered at a dose of about 15, about 20 or about 40 mg/kg,
(d) is administered at a dose of about 6 mg/kg, and
(e) is administered at a dose of about 75 mg/m².

4. The method of claim 3, wherein, in the first cycle, one or more of (a), (d), and (e) is administered at a loading dose that is greater than the corresponding doses of one or more of (a), (d), and (e) administered in each subsequent cycle.

5. The method of any one of claims 1-4, wherein (a) comprises a polypeptide having an amino acid sequence as set forth in SEQ ID NO:1.

6. The method of claim 1 or claim 2, wherein, during each week of each cycle, order of administration is: (b) is administered first, (c) is administered second, (d) is administered third, and (a) is administered fourth.

7. The method of claim 4 or claim 5, wherein, during each three week cycle, order of administration is: (e) is administered first, (d) is administered second, and (a) is administered third.

8. The method of any one of claims 1-7, wherein the patient is pretreated prior to administration of the taxane with at least one dose of an agent that prevents taxane hypersensitivity.

9. The method of claim 8, wherein the at least one dose of the agent that prevents hypersensitivity is two 20 mg doses of dexamethasone; one dose of 50 mg of diphenhydramine; one dose of 300 mg of cimetidine; or one dose of 50 mg of ranitidine.

10. The method of any one of claims 1-9, wherein the treatment comprises at least 4 cycles.

11. The method of any one of claims 1-10, wherein the treatment produces at least one therapeutic effect selected from the group consisting of reduction in size of a tumor, reduction in number of metastatic lesions over time, complete response, partial response, stable disease, increase in overall response rate, increase in overall survival, and an increase in progression-free survival.

12. The method of any one of claims 1-11, wherein the patient is additionally treated with G-CSF.

13. The method of any one of claims 1-12, wherein the cancer is a solid tumor.

14. The method of claim 13 wherein the tumor is a HER2-FISH positive tumor.

15. The method of claim 13, wherein the tumor is a HER2 2+ tumor.

16. The method of claim 13, wherein the tumor is a HER2 3+ tumor.

17. The method of claim 13, wherein the tumor is a HER2 2+, HER2 FISH-negative tumor.

18. The method of claim 13, wherein the cancer is a breast, esophageal, gastric, gastro-esophageal junction, bladder, ovarian, endometrial, colorectal or non-small cell lung cancer.

19. The method of any of claims 1-18, wherein the patient has undergone at least six cycles of treatment, and wherein a maintenance dose of trastuzumab, and optionally a bispecific anti-ErbB2/anti-ErbB3 antibody, is administered to the patient.

20. A container comprising an effective amount of a bispecific anti-ErbB2/anti-ErbB3 antibody, and instructions for using the bispecific anti-ErbB2/anti-ErbB3 antibody according to the method of any one of claims 1-19.

21. The container of claim 20, said container comprising at least 250 mg of the bispecific antibody.

22. The container of claim 20 or claim 21, said container further comprising an effective amount of one or more of docetaxel, lapatinib, paclitaxel, and trastuzumab.

23. A combination for use in treating a cancer in a human patient, the combination comprising a clinically proven safe and effective amount of (a) a bispecific anti-ErbB2/anti-ErbB3 antibody, (b) lapatinib, (c) paclitaxel and (d) trastuzumab.

24. A combination for use in treating a cancer in a human patient, the combination comprising a clinically proven safe and effective amount of (a) a bispecific anti-ErbB2/anti-ErbB3 antibody, (e) docetaxel and (d) trastuzumab.

25. An antibody for co-administration with lapatinib, a taxane that is a paclitaxel, and trastuzumab in at least one cycle, wherein the cycle is a period of four weeks, and wherein during each of the four weeks of each cycle the antibody is administered at a weekly starting dose of about 5, about 10, about 20, about 30, about 40, or about 50 mg/kg, the lapatinib is administered at a daily dose of about 750 or about 1000 mg, the paclitaxel is administered at a weekly dose of about 80 mg/m², and the trastuzumab is administered at a weekly dose of about 2 mg/kg and wherein the antibody is a bispecific anti-ErbB2/anti-ErbB3 antibody comprising a polypeptide having an amino acid sequence as set forth in SEQ ID NO:1.

26. An antibody for co-administration with docetaxel and trastuzumab in at least one cycle, wherein the cycle is a period of three weeks, and wherein once during each cycle the bispecific anti-ErbB2/anti-ErbB3 antibody is administered at a dose of about 15, about 20, about 30, about 40, or about 50 mg/kg, the docetaxel is administered at a dose of about 75 mg/m², and the trastuzumab is administered at a dose of about 6 mg/kg, wherein the antibody is a bispecific anti-ErbB2/anti-ErbB3 antibody comprising a polypeptide having an amino acid sequence as set forth in SEQ ID NO:1.

27. The method of any of claims 1-19, wherein the bispecific anti-ErbB2/anti-ErbB3 antibody is administered using a low-protein-binding 0.22 micrometer in-line filter.

28. The method of any of claims 1-19, wherein the patient is pretreated with one or more of corticosteroids, diphenhydramine, and 1-12 antagonists.

29. The method of any of claims 1-19, wherein a loading dose of the bispecific anti-ErbB2/anti-ErbB3 antibody is administered during cycle 1.

30. The method of claim 29, wherein the loading dose is 25 mg/kg.
